(19) United States
(12) Patent Application Publication
(10) Pub. No.: US 2012/0209126 A1
(43) Pub. Date: Aug. 16, 2012

(54) METHOD AND SYSTEM FOR DETECTING CARDIAC ARRHYTHMIA

(75) Inventors: Yariv Avraham Amos, Hadera (IL); Gil Kaminski, Givat Shmuel (IL)

(73) Assignee: Widemed Ltd., Herzlia (IL)

(21) Appl. No.: 13/502,827

(22) PCT Filed: Oct. 20, 2010
(86) PCT No.: PCT/IL2010/000860

§ 371 Date: Apr. 19, 2012

Related U.S. Application Data
(60) Provisional application No. 61/253,110, filed on Oct. 20, 2009.
(60) Provisional application No. 61/254,704, filed on Oct. 25, 2009.

(51) Int. Cl. A61B 5/0452; A61B 6/00; A61B 5/021; A61B 5/02

(52) U.S. Cl. 600/479; 600/515; 600/513; 600/485

(57) ABSTRACT

A method of analyzing physiological data indicative of myocardial activity is disclosed. The method comprises: identifying in the data a set of N features, each corresponding to a ventricular depolarization, and calculating M time-intervals for each ventricular depolarization feature, thereby providing a vector of N*M time-intervals. The method further comprises fitting the vector to a power density function of time-intervals, and determining possible cardiac arrhythmia based on statistical parameters characterizing the function.

### RELATED APPLICATIONS

This application claims the benefit of priority from U.S. Patent Application Nos. 61/253,110 filed Oct. 20, 2009, and 61/254,704 filed Oct. 25, 2009, the contents of which are hereby incorporated by reference as if fully set forth herein.

### FIELD AND BACKGROUND OF THE INVENTION

The present invention, in some embodiments thereof, relates to physiological monitoring and diagnosis and, more particularly, but not exclusively, to a method and system for detecting cardiac arrhythmia. Some embodiments of the present invention relate to classification of heart beats.

Cardiac arrhythmias can generally divide into life-threatening arrhythmias such as ventricular fibrillation and tachycardia, and non-life-threatening arrhythmias that are not imminently life threatening, such as atrial fibrillation, atrial flutter, ventricular bigeminy and ventricular trigeminy. By way of background, non-life-threatening arrhythmias will now be explained.

In cardiac practice, heartbeats are named according to the initial source of the heartbeat. The normal beating of the heart is known as “sinus rhythm”, because the normal heart beat is initiated by a small area of specialized muscle in the atria referred to as the sinoatrial (SA) node (or more commonly, the “sinus node”). When electrical activity is spontaneously generated by the sinus node, the electrical impulse is propagated throughout both the right atrium and left atrium, stimulating the myocardium of the atria to contract. When the atria contract, blood is pumped from the atria of the heart to the lungs and then back into the ventricles.

On an electrocardiogram (ECG), the P-wave represents the electrical potential generated by atrial muscle cell depolarization as the heart's atrial chambers contract. The spread of electrical activity through the ventricular myocardium causes the ventricles of the heart to contract. When the ventricles contract, the blood in the ventricles is pumped at high pressure around the body (and eventually back to the atria). The QRS complex of the ECG represents the electrical potential generated by ventricular muscle cell depolarization as the heart's ventricular chambers contract.

The atrioventricular (AV) node is a specialized section of the myocardium located between the atria and the ventricles. The AV node functions as a critical delay in the conduction system. In order for the heart to work well, the heart must first pump blood from the atria to the ventricles (via the lungs, where the blood becomes oxygenated). Once this occurs, the ventricles then pump the oxygenated blood throughout the body. The AV delay allows the atria to fill the ventricles with blood before the ventricles are pumped. If the ventricles are pumped prior to being filled with blood from the lungs via the atria, the ventricular pump action would oppose the movement of blood from atria to ventricles and reduce the pressure of the blood moving from the ventricles to the rest of the body. On the ECG, the delay in the AV node is observed as the segment between the P wave and the R wave.

The last event of the cardiac cycle is the repolarization of the ventricles. This event is manifested by the T-wave of the ECG. The T-wave represents the electrical potential generated as the ventricles of the heart recover (or repolarize) from a state of depolarization after the QRS complex has occurred. In principle, there is an equivalent repolarization wave for the P-wave, occurring during the PR segment and traversing somewhat into the QRS complex. However, from a surface ECG, this repolarization signal is typically too small to be seen.

There are several generic features of sinus rhythm that serve as hallmarks for comparison with normal ECGs. These include the PR-interval, the ST-segment and the QT interval. The PR-interval is typically measured from the beginning of the P-wave to the beginning of the QRS complex, the ST-segment is typically measured from the end of the QRS complex to the beginning of the T-wave, and the QT-interval is measured from the beginning of the QRS complex to the end of the T-wave.

The distance between the R waves of two successive cardiac cycles is known as the RR interval. While one would ideally measure the “ventricular rate” as the QQ interval (typically the interval from QRS onset to the next QRS onset), in practice, the RR interval is used as the measurement of ventricular rate, due to the practical difficulty of reliably measuring the small, inconsistently sized and inconsistently occurring Q-wave.

In a heart in sinus rhythm, the five distinct waves of the ECG (P, Q, R, S and T) as well as the various segments and intervals (e.g., PR, ST, QT, and RR) occur in a specific order with an expected range of relative sizes. While there is a significant range within which variations in rhythm that are considered normal, anything that deviates from sinus rhythm by more than a certain amount may be indicative of a heart condition.

A heartbeat having an atrial origin, excluding the sinus node, is collectively referred to as an “atrial ectopic”. Were this heartbeat occurs faster than the current sinus rate, it is termed a “premature atrial contraction” (PAC), and were it occurs slower than the current sinus rate it is termed an “atrial escape beat”. Similarly, for ventricular activity, the collective term “ventricular ectopic,” and the specific terms “premature ventricular contraction” (“PVC”), and “ventricular escape beat” are used.

Almost any area of the heart can generate a heartbeat as a back-up mechanism for when the sinus node does not start a heartbeat when it should. Escape ectopics are, therefore, a manifestation of the back-up mechanism working correctly and are thus not themselves a problem but rather indicate that a problem has occurred with the sinus node. However, premature contractions occur before the sinus node and override the correct sinus beat, thus indicating a problem with the area of the heart that prematurely generated an erroneous back-up beat.

Most people spend most of their time in sinus rhythm, with some infrequent ectopics occurring. When ectopics become frequent, it is usually caused by a specific part of the heart causing a problem. For example, a specific area of the heart may be implicated if a particular PVC becomes common, sometimes occurring in lengthy patterns. An example of such pattern is a ventricular bigeminy, which is a repetitive pattern including a sinus beat followed by a PVC. Another example is known is a ventricular trigeminy, which is a repetitive pattern including two sinus beats followed by a PVC, or two PVCs followed by a sinus beat.

Atrial fibrillation and atrial flutter (collectively referred to as AF) are two related types of cardiac arrhythmia where the entire atrium (rather than just a specific problematic area) starts to generate electrical impulses that can initiate a heartbeat. Effectively, AF is caused by many different atrial ectopics, all in competition with each other, overwhelming the sinus node. Because the area of the heart that generates the next heartbeat is not fixed, the heart rate of the next heartbeat is also not fixed and thus a highly chaotic sequence of heartbeats is observed. In addition, several P-waves per QRS complex are observed, as the ventricles cannot respond to every P-wave the atria generate. As the P-waves originate from different parts of the atria, their shapes are not constant, so the collection of high-rate P-waves between QRS complexes in AF can often resemble little more than a messy line on an ECG. Thus, in AF, the electrical impulses that are normally generated by the SA node are replaced by disorganized activity in the atria. In the case of atrial flutter, some level of organization can sometimes occur in the atria, with the multiple-P-waves starting to look like a train of saw-tooth waves at a very high atrial rate.

Known in the art are many techniques which take advantage of high RR variability for detecting arrhythmias. Representative examples include U.S. Pat. Nos. 7,794,406, 6,490,479, 6,871,089, 6,519,490, 6,922,584, 7,031,765, 7,120,485, 7,194,300 and 6,597,943; U.S. Published Application No. 20090012412; Tateno et al. “A Method for Detection of Atrial Fibrillation Using RR intervals,” Computer In Cardiology, vol. 27 pp. 391-394; Moody et al., “A New Method for Detecting Atrial Fibrillation using R-R intervals,” Computer in Cardiology, pp. 227-230, 1983; Artis et al., “Detection of atrial fibrillation using artificial neural networks,” Computers in Cardiology pp. 173-176; Logan B T. Healey J., “Robust Detection of Atrial Fibrillation for a Long Term Telemonitoring System,” Computer In Cardiology, pp. 25-28, 2005; and Zurro et al., “Detection of Atrial Persistent Rhythm Based on P-Wave Recognition and RR Interval Variability,” Computer In Cardiolog, pp. 185-188, 1995.

By way of example, U.S. Pat. No. 7,794,406 discloses a technique in which waveforms that are indicative of a cycle of the blood flow are extracted from a photoplethysmograph (PPG) signal. An aberrant waveform is then identified among the extracted waveforms, and its shape is analyzed to identify an irregularity in the heart rhythm of the patient. The time of occurrence of the irregularity is processes to diagnose a pathological cardiac condition of the patient. U.S. Published Application No. 20090012412 discloses a method in which a sequence of pulse beats are detected to provide a succession of time intervals corresponding to the sequence of pulse beats. Thereafter, the mean of the succession of time intervals is ascertained and lower and upper boundary values each as a respective percent of the mean are determined. The mean is recalculated, and a standard deviation based on the succession of time intervals that are between the upper and lower boundary values is calculated. A possible atrial fibrillation is determined based upon a ratio between the standard deviation and the recalculated mean.

### SUMMARY OF THE INVENTION

According to an aspect of some embodiments of the present invention there is provided a method of analyzing physiological data indicative of myocardial activity. The method comprises: identifying in the data a set of N features, each corresponding to a ventricular depolarization, where N is an integer larger than 1. The method also comprises calculating M time-intervals for each ventricular depolarization feature, thereby providing a vector of N*M time-intervals, where M is an integer larger than 1. The method further comprises fitting the vector to a power density function ƒ of time-intervals, and determining possible cardiac arrhythmia based on statistical parameters characterizing the power density function ƒ.

According to some embodiments of the invention the parameters comprise at least one of a time-interval separation parameter, and a variance of ratios between widths and centers characterizing the power density function of the time-intervals.

According to some embodiments of the invention the method comprises calculating, for each time-interval, M differences between time-intervals, thereby providing a vector drrs of N*M time-interval differences, wherein the possible cardiac arrhythmia is determined based, at least in part, on a standard deviation of the vector drrs.

According to some embodiments of the invention the method comprises, for each ventricular depolarization feature, calculating a relative blood volume value, thereby providing a vector rbvs of N relative blood volume values. In some embodiments, the method calculates for each ventricular depolarization feature K relative blood volume values. In these embodiments the vector rbvs has N*K relative blood volume values. According to some embodiments of the invention the method comprises fitting the vector rbvs to a power density function g of relative blood volume values, wherein the determination of possible cardiac arrhythmia is also based on an additional set of statistical parameters characterizing the power density function g.

According to some embodiments of the invention the method comprises, applying to the statistical parameters a thresholding procedure having a set of criteria, wherein fulfillment of the criteria by the statistical parameters indicates possible arrhythmia among the N ventricular depolarization features.

According to some embodiments of the invention if the criteria are fulfilled, then the method further comprises: calculating a second order localized mixture model within a space spanned by the statistical parameters; applying a set of criteria to the second order localized mixture model so as to provide a model classified as normal and a model classified as abnormal; and, for a given segment of the data, determining the likelihood that the segment is abnormal by calculating similarities of the segment to the first and the second models.

According to some embodiments of the invention the method comprises identifying data features corresponding to premature heartbeats.

According to an aspect of some embodiments of the present invention there is provided a computer-readable medium having stored thereon a computer program, wherein the computer program comprising code means that when executed by a data processing system carry out at least part of the method described herein.

According to an aspect of some embodiments of the present invention there is provided a system for analyzing physiological data indicative of myocardial activity. The system comprises a data processing system configured for: identifying in the data a set of N features, each corresponding to a ventricular depolarization, where N is an integer larger than 1. The data processing system is also configured for calculating M time-intervals for each ventricular depolarization feature, thereby providing a vector of N*M time-intervals, where M is an integer larger than 1. The data processing system is further configured for fitting the vector to a power density function ƒ of time-intervals, and determining possible cardiac arrhythmia based on statistical parameters characterizing the power density function ƒ.

According to some embodiments of the invention the power density function comprises a sum of localized functions.

According to some embodiments of the invention an expectation and variance of each of the localized functions are independent of a time-point of a respective ventricular depolarization feature.

According to some embodiments of the invention the power density function represents a degenerated mixture model.

According to some embodiments of the invention the degenerated mixture model is a degenerated Gaussian mixture model.

According to some embodiments of the invention the parameters comprise at least one of a time-interval separation parameter, and a variance of ratios between widths and centers characterizing the power density function of the time-intervals.

According to some embodiments of the invention the data processing system is configured for calculating, for each time-interval, M differences between time-intervals, thereby providing a vector drrs of N*M time-interval differences, wherein the possible cardiac arrhythmia is determined based, at least in part, on a standard deviation of the vector drrs.

According to some embodiments of the invention the data processing system is configured for calculating, for each ventricular depolarization feature, a relative blood volume value, thereby providing a vector rbvs of N relative blood volume values. In some embodiments, the data processing system is configured for calculating for each ventricular depolarization feature K relative blood volume values. In these embodiments the vector rbvs has N*K relative blood volume values. According to some embodiments of the invention the data processing system is configured for fitting the vector rbvs to a power density function g of relative blood volume values, wherein the determination of possible cardiac arrhythmia is also based on an additional set of statistical parameters characterizing the power density function g.

According to some embodiments of the invention the additional set of parameters comprises at least one of a relative blood volume separation parameter, and a variance of ratios between widths and centers characterizing the power density function of the relative blood volume values.

According to some embodiments of the invention the data processing system is configured for applying to the statistical parameters a thresholding procedure having a set of criteria, wherein fulfillment of the criteria by the statistical parameters indicates possible arrhythmia among the N ventricular depolarization features.

According to some embodiments of the invention the data processing system is configured for: calculating a second order localized mixture model within a space spanned by the statistical parameters; applying a set of criteria to the second order localized mixture model so as to provide a model classified as normal and a model classified as abnormal; for a given segment of the data, determining the likelihood that the segment is abnormal by calculating similarities of the segment to the first and the second models.

According to some embodiments of the invention each of the localized mixture models is independently a Gaussian mixture model.

According to some embodiments of the invention the data processing system is configured for identifying data features corresponding to premature heartbeats.

According to some embodiments of the invention the data comprises photoplethysmograph data.

According to some embodiments of the invention the data comprises electrocardiogram data.

According to some embodiments of the invention the data comprises continuous blood pressure data.

According to some embodiments of the invention the system comprises at least one sensor for receiving a signal indicative of the myocardial activity, and circuitry for generating the physiological data responsively to the signal.

According to some embodiments of the invention the sensor(s) comprises a photoplethysmograph. According to some embodiments of the invention the sensor(s) comprises a single lead a photoplethysmograph.

According to some embodiments of the invention the sensor(s) comprises at least one electrocardiogram lead.

According to some embodiments of the invention the sensor(s) comprises at least one blood pressure sensor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram describing a method suitable for analyzing physiological data indicative of myocardial activity according to various exemplary embodiments of the present invention;

FIG. 2 is a schematic illustration of a system 20 for analyzing physiological data indicative of myocardial activity, according to some embodiments of the present invention;

FIGS. 3A-D show 10 seconds of normal heart rhythm;

FIGS. 4A-D show 10 second of atrial fibrillation rhythm;

FIGS. 5A-C show normalized histograms of three statistical parameters calculated according to some embodiments of the present invention;

FIGS. 6A-C show the results of an adaptive classification procedure, employed according to some embodiments of the present invention;

FIG. 7 illustrates a procedure for defining time-intervals for a series of QRS complexes, according to some embodiments of the present invention;

FIGS. 8A-B shows a representative example of a premature ventricular contraction beat as it is manifested on an ECG (FIG. 8A) and a PPG (FIG. 8B);

FIGS. 9A-B show a premature atrial contraction as manifested on an ECG (FIG. 9A) and a PPG (FIG. 9B); and

FIGS. 10A-B show bigeminy rhythm as manifested on an ECG (FIG. 10A) and a PPG (FIG. 10B).

### DESCRIPTION OF SPECIFIC EMBODIMENTS OF THE INVENTION

The present invention, in some embodiments thereof, relates to physiological monitoring and diagnosis and, more particularly, but not exclusively, to a method and system for detecting cardiac arrhythmia. Some embodiments of the present invention relate to classification of heart beats.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present embodiments are concerned with method and system for analyzing physiological data indicative of myocardial activity to determine possible cardiac arrhythmia and, in some embodiments, to classify individual segments of the data according to the likelihood of cardiac arrhythmia. At least part of the processing can be implemented by a data processing system, e.g., a computer, configured for receiving the data and executing the operations described below.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

Computer programs implementing the method of the present embodiments can commonly be distributed to users on a distribution medium such as, but not limited to, a floppy disk, a CD-ROM, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

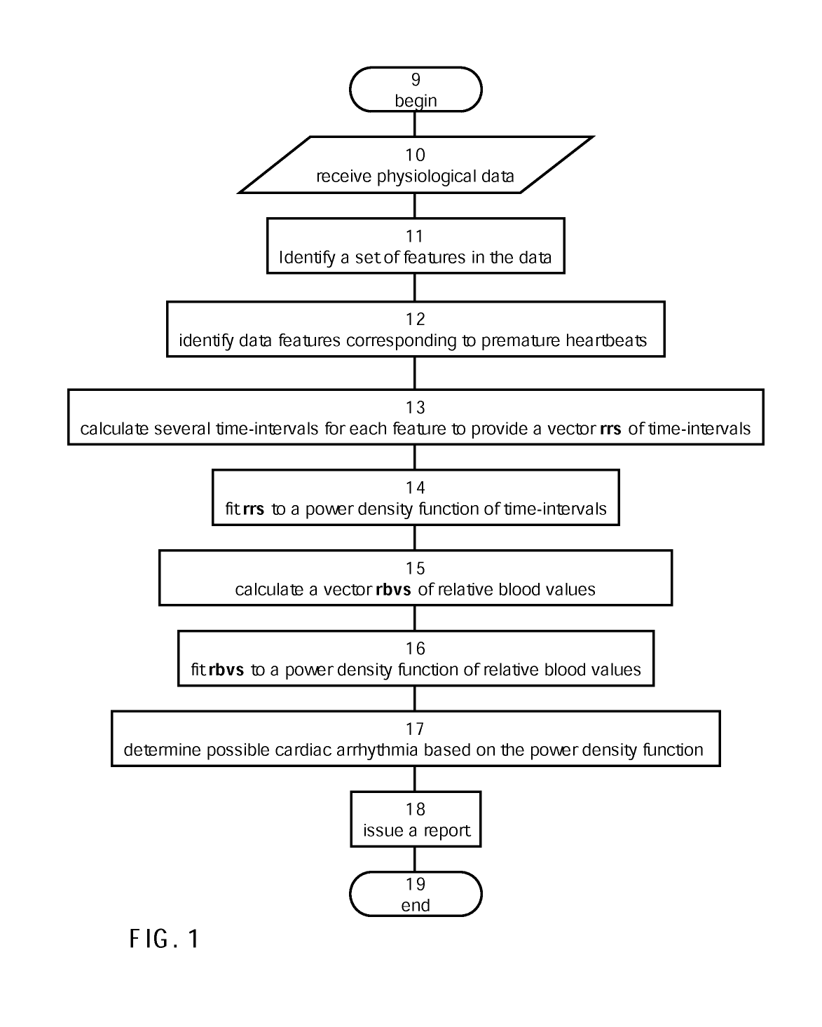

Referring now to the drawings, FIG. 1 is a flowchart diagram describing the method according various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 9 and continues to data block 10 at which the physiological data to be analyzed are received. Typically, but not necessarily the data comprise photoplethysmograph (PPG) data or ECG data or continuous blood pressure data. Other types of physiological data are not excluded from the scope of the present invention provided that the data are indicative of myocardial activity. In some embodiments of the present invention the data comprise only one type of data. For example, the data can be PPG data, in which case the data is devoid of ECG data, devoid of continuous blood pressure data and devoid of any other type of data; alternatively, the data can be ECG data, in which case the data is devoid of PPG data, devoid of continuous blood pressure data and devoid of any other type of data; still alternatively, the data can be continuous blood pressure data, in which case the data is devoid of ECG data, devoid of PPG data and devoid of any other type of data.

Also contemplated, are embodiments in which several types of data are used. For example, the data can include both PPG and ECG data. In various exemplary embodiments of the invention each type of data is analyzed independently of any other data.

The data to be analyzed preferably comprises digital data. Digital data is typically generated by appropriate circuitry which receives a signal indicative of myocardial activity (e.g., from a PPG sensor and/or one or more ECG leads and/or one or more sensors arranged to continues receive signals indicative of blood pressure) and converts the signal to digital data. Such types of digitizer circuitries are known in the art.

The method continues to 11 at which a set of features is identifying in the data, each feature corresponding to a ventricular depolarization. Techniques for automatic identification of ventricular depolarization features are known in the art and found, e.g., in a book by Kay, S. M., “Fundamental of Statistical Signal Processing: Detection Theory,” published by Prentice Hall Inc., 1998.

When the data include ECG data, the ventricular depolarization feature can correspond to the QRS complex of the ECG. However, it is not necessary to display or render a graphical representation of the data in order to identify such feature. For example, a filter, e.g., a band pass filter with cutoff frequencies of about 0.2 Hz and about 30 Hz, can be applied to the ECG data, and the filtered data can thereafter be subjected to a numerical derivative operation. A moving average can them be employed for detecting peaks in the data. The peak height, location and maximum derivative can be used to classify each detected peak as an R peak of a QRS complex or other peak.

When the data include PPG data, a filter, e.g., a band pass filter with cutoff frequencies of about 0.05 Hz and about 5 Hz, can be applied to the PPG data, so as to suppress very low frequency and high frequency noise. Optionally and preferably systematic shifts are removed from the filtered PPG data. Such types of operations are known in the art and are oftentimes referred to in the literature as “de-trending”. For example, in some embodiments, the data are fitted to some predetermined line (e.g., a straight line or a segmented straight line), and the fitted line is subtracted from the data. Removal of systematic shifts is advantageous since it allows further analysis of fluctuations in the data about the systematic shift. The ventricular depolarization features can be identified by segmenting the PPG data into pulses and marking each or some of the pulses as a ventricular depolarization feature. Possible criteria for marking a pulse as a ventricular depolarization feature, without limitation, a duration from a previous peak and a ratio between successive peaks. Optionally and preferably, the normalized area of each identified pulse is calculated. In some embodiments of the present invention the normalized area is used as a value indicative of relative blood volume. Other techniques for identifying ventricular depolarization features are also contemplated. For example, U.S. Pat. No. 5,588,425 describes the use of a pulse oximeter in validating the heart rate and/or R-R intervals. U.S. Pat. No. 7,001,337 describes a method for obtaining heart rate, heart rate variability and blood volume variability from PPG. The disclosures of the above-mentioned patents are incorporated by reference as if fully set forth herein.

When the data include continuous blood pressure data, the procedure for identifying ventricular depolarization features can be similar to one of the procedures described above regarding PPG data.

The number of ventricular depolarization features identified at 11 is denoted hereinafter by N, where N is an integer larger than 1.

In some embodiments of the present invention the method continues to 12 at which the method identifies data features corresponding to premature heartbeats. Once the premature heartbeats are identified, they are preferably marked and excluded from further analysis. Alternatively, isolated premature beats are marked but not removed, wherein one or more patterns, such as but not limited to, ventricular bigeminy pattern and/or ventricular trigeminy ventricular is identified and excluded from further analysis. A preferred technique for identifying premature heartbeats is described hereinunder.

The method continues to 13 at which several time-intervals are calculated for each ventricular depolarization feature. The number of time-intervals that are calculated for each feature is denoted hereinafter by M, where M is an integer larger than 1. The M calculated time-intervals per feature can be collected as the components of a vector denoted rr[n] of dimension M, where n is an index representing the time-point of the respective ventricular depolarization feature (n=1, . . . , N). Throughout this specification, vector quantities are denoted by bold letters. A preferred expression for the vector rr[n] is:

rr[n]=[rr₁[n],rr₂[n], . . . ,rr_{M}[n]] (EQ. 1)

where rrⱼ[n]=r[n]−r[n−j], j=1, . . . ,n−1, and r[n] denotes the location of the nth feature.

In embodiments in which isolated premature beats are marked but not removed, the instantaneous series of time-intervals is smoothed in order to remove the abnormality in the rhythm.

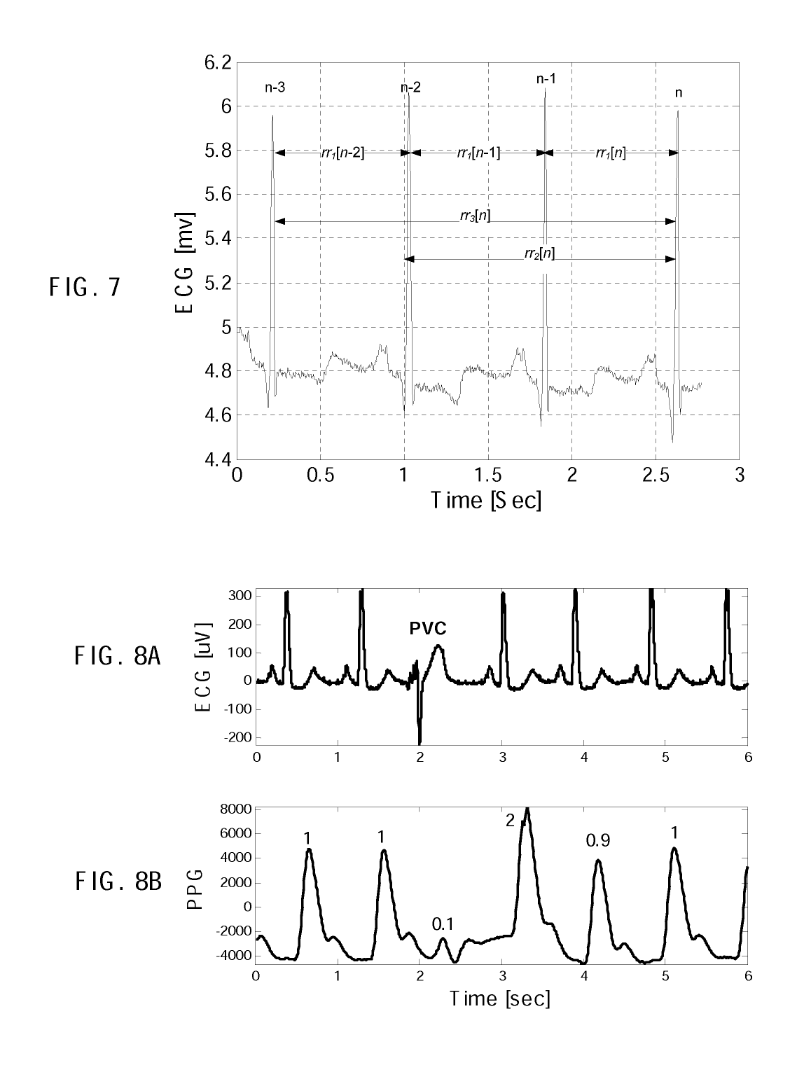

The time-intervals rrⱼ[n] can be better understood with reference to FIG. 7. Shown in FIG. 7 are 4 normal QRS complexes occurring at the following approximate time points: 0.2 s, 1.05 s, 1.75 s and 2.6 s. These complexes are enumerated n−3, n−2, n−1 and n, respectively. The time-interval between complex n and complex n−1 is denoted rr₁[n], the time-interval between complex n and complex n−2 is denoted rr₂[n], the time-interval between complex n and complex n−3 is denoted rr₃[n], and the time-interval between complex n and complex n−4 is denoted rr₄[n]. Thus, FIG. 7 corresponds to EQ. 1 with M=4. Notice that rrⱼ[n] is a component of a vector, namely a number. Notice further that for 1≦j≦n−1, rrⱼ[n] satisfies:

rrⱼ[n]=rr₁[n]+rr₁[n−1]+ . . . +rr₁[n−j+1] (EQ. 2)

(to this end see FIG. 7 in which rr₁[n]+rr₁[n−1]+rr₁[n−2]=rr₃[n]).

The N vectors that are calculated at 13 are optionally and preferably concatenated into a vector denoted hereinafter by rrs. Formally, rrs can be written as:

rrs=[rr[1],rr[2], . . . ,rr[N]]. (EQ. 3)

It is appreciated that the dimension of the vector rrs is N*M where the asterisk denotes arithmetic multiplication. A typical value for N is from about 5 to about 60 ventricular depolarization beats in a given segment, and a typical value of M is from about 2 to about 20. Thus, the number of components of vector rrs is typically from about 10 to about 1200 samples.

The method continues to 14 at which the vector rrs is fitted to a power density function ƒ of a variable x representing time-intervals. In various exemplary embodiments of the invention the power density function comprises a sum of localized functions. Representative examples of localized functions include, without limitation, Gaussian functions, Lorentzian functions, hyperbolic secant functions (also known as sech), logistic distributions, multinomial distributions, Poisson distributions, multivariate Gaussian distributions, and the like. Also contemplated are embodiments in which each localized function is represented as a series or an integral of other functions. For example, a localized function can be a Fourier transform, a wavelet transform and the like.

In some embodiments of the present invention the expectation value and variance of each of the localized functions are independent of a time-point of a respective ventricular depolarization feature. In terms of the variables and indices defined above, the expectation value and variance of each of the localized function do not depend on the index n. For example, when the localized functions are Gaussians, these embodiments can be expressed as follows:

E[rrⱼ[n]]=jλ,var[rrⱼ[n]]=jσ², (EQ.4)

where E represent the expectation value, var represent the variance, and λ and σ are, respectively, the center and width of the first Gaussian. In these embodiments the power density function can have the form:

$$f ( x \vert λ , σ ) = {\sum }_{i = 1}^{M} \frac{1}{M \sqrt{2 {πσ}^{2}}} {e}^{- \frac{{( x - i λ )}^{2}}{2 {σ}^{2}}} . \qquad ( EQ . 5 )$$

In alternative embodiments, the power density function represents a degenerated mixture model, such as, but not limited to, a degenerated Gaussian mixture model (GMM). Due to the unique property of the rrs (see EQ. 4) the GMM is optionally and preferably a degenerated GMM (D-GMM).

In these embodiments, the expectation value and variance of each of the localized functions vary with n. Specifically, in these embodiments the center and width of each localized function is independent of the center and width of other localized function. For the ith localized function in a degenerated mixture model, is the center is denoted λᵢ and the width is denoted σᵢ. It is convenient to parameterize the fluctuation around the linear trend of λᵢ using a center-fluctuation parameter Δλᵢ, e.g., λᵢ=iλ+Δλᵢ. For example, when a D-GMM is employed, the power density function can have the form:

$$f ( x \vert θ ) = {\sum }_{i = 1}^{M} \frac{1}{M \sqrt{2 {πσ}_{i}^{2}}} {e}^{- \frac{( x - i λ - {Δλ}_{i} )}{2 {σ}_{i}^{2}}} \qquad ( EQ . 6 )$$

where θ is a vector of dimension 2M+1 defined as θ=[λ, σᵢ, Δλᵢ]ᵢ₌₁^{M}.

In some embodiments of the present invention, the method continues to 15 at which for each ventricular depolarization feature, the method calculates relative blood volume values to provide a vector rbvs of relative blood volume values. A relative blood volume value can be obtained, for example, by calculating the normalized area of the peak corresponding to the respective feature. This embodiment is particularly useful when the data include PPG data.

A preferred procedure for calculating the vector rbvs is as follows. First, N vectors or scalars each of dimension K are calculated for the nth feature as follows:

rbv[n]=[rbv₁[n],rbv₂[n], . . . ,rbv_{K}[n]] (EQ. 7)

where:

rbvⱼ[n]=[rbv[n]+rbv[n−1]+ . . . +rbv[n−j+1]],j=1 . . . K, (EQ. 8)

and rbv[n] is the relative blood volume of the nth feature. K is a positive integer. In various exemplary embodiments of the invention K is larger than 1 (e.g., 5, 10 or more), but embodiments in which K=1, namely embodiments in which rbv[n] is a scalar, are not excluded from the scope of the present invention.

Thereafter, the vector rvbs is constructed by concatenation:

rbvs=[rbv[1],rbv[2], . . . ,rbv[N]]. (EQ. 9)

It is appreciated that rbvs is a vector of dimension N*K.

It was found by the present inventors that in case of a normal rhythm the vector rbvs follows the aforementioned D-GMM distribution where the first Gaussian has a mean value of 1 (the relative values are normalize such that the typical value is one), hence

E[rbvⱼ[n]]=j,var[rbvⱼ[n]]=jv², (EQ.10)

where v is the standard deviation.

Once the vector rbvs is obtained, the method proceeds to 16 at which rbvs is fitted to a power density function g of a variable y representing blood volume values. In various exemplary embodiments of the invention the power density function comprises a sum of localized functions. Any of the aforementioned localized functions can be used, including series or integrals of other function.

The expectation value and variance of each of the localized functions can be independent of or vary with the time-point of the respective ventricular depolarization feature. A representative example of the power density function for the former case is:

$$g ( y \vert v ) = {\sum }_{i = 1}^{K} \frac{1}{K \sqrt{2 π {v}^{2}}} {e}^{- \frac{{( y - i )}^{2}}{2 {v}^{2}}} , \qquad ( EQ . 11 )$$

and a representative example of the power density function for the latter case is:

$$g ( y \vert ξ ) = {\sum }_{i = 1}^{K} \frac{1}{K \sqrt{2 {πυ}_{i}^{2}}} {e}^{- \frac{{( y - i + {Δμ}_{i} )}^{2}}{2 \cdot {υ}_{i}^{2}}} \qquad ( EQ . 12 )$$

where ξ is a vector of dimension 2K defined as ξ=[vᵢ, Δμᵢ]ᵢ₌₁^{K}.

The method continues to 17 at which a possible cardiac arrhythmia is determined based on statistical parameters characterizing the power density function ƒ and/or g. Several statistical parameters are contemplated by the present inventors. In some embodiments of the present invention the statistical parameters are those which define the respective power density function. For example, when the power density function is such that the expectation value, E, and variance, var, of each localized function are independent of n, such parameters include E and var. In the alternative embodiments, more parameters define the power density function. For example, when the variance and expectation value vary with n, the statistical parameters of ƒ can include all 2M+1 components of the vector θ defined above, and the statistical parameters of g can include the 2K components of the vector ξ.

The present inventors also contemplate use of one or more of the above parameters for calculating other statistical parameters. For example, in some embodiments of the present invention the statistical parameters include one or more time-interval separation parameters. This type of parameters characterizes the amount of overlaps among the localized functions of ƒ or g, and is particularly useful when var and E vary with n. The separation parameters can be calculated by averaging over sum of widths and differences between centers of the respective localized function. A preferred expression for a separation parameter is:

$$\begin{matrix}{S}_{M}^{rr} = \frac{1}{M - 1} {\sum }_{i = 1}^{M - 1} ( {λ}_{m + 1} - {σ}_{m + 1} ) - ( {λ}_{m} + {σ}_{m} ) , & ( EQ . 13 ) \\ \begin{matrix}{S}_{K}^{rbv} = \frac{1}{K - 1} {\sum }_{k = 1}^{K - 1} ( k + 1 + {Δμ}_{k + 1} - {v}_{k + 1} ) - ( k + {Δμ}_{k} + {v}_{k} ) \\ = \frac{1}{K - 1} {\sum }_{k = 1}^{K - 1} ( 1 + {Δμ}_{k + 1} - {v}_{k + 1} ) - ( {Δμ}_{k} + {v}_{k} )\end{matrix} & ( EQ . 14 )\end{matrix}$$

EQ. 13 and EQ. 14 represent the separation parameters which characterize overlaps among the localized functions of ƒ and g, respectively. It is appreciated that larger value of the separation parameters indicates less overlap among the localized functions.

In some embodiments of the present invention the statistical parameters include one or more variances of a ratio between a width and a center of the localized functions. This type of statistical parameters characterizes the amount of non-uniformity in the dispersions of the localized functions of ƒ or g. The ratio between the width and the center of the ith localized function is denoted Cᵢ. For example, when the localized functions are Gaussians, Cᵢ is the coefficient of variation of the respective Gaussian. In these embodiments Cᵢ is given by Cᵢ=σᵢ/λᵢ for the power density function ƒ and by Cᵢ=vᵢ/(1+Δμᵢ) for the power density function g.

A preferred expression for the variance of C is:

$$\begin{matrix}{VC}_{M}^{rr} = \frac{1}{M} {\sum }_{m = 1}^{M} ( {C}_{m}^{2} ) - {( \frac{1}{M} {\sum }_{m = 1}^{M} {C}_{m} )}^{2} & ( EQ . 15 ) \\ {VC}_{K}^{rbv} = \frac{1}{K} {\sum }_{k = 1}^{K} ( {C}_{k}^{2} ) - {( \frac{1}{K} {\sum }_{k = 1}^{K} {C}_{k} )}^{2} . & ( EQ . 16 )\end{matrix}$$

Also contemplated, are statistical parameters which are not extracted from the power density functions themselves. For example, in some embodiments of the present invention the method calculates, for each time-interval, M differences between time-intervals to provide a vector drrs of N*M time-interval differences. In these embodiments, the possible cardiac arrhythmia is determined based, at least in part, on a standard deviation std(drrs) of this vector.

A typical expression for the vector drrs is:

drrs=[drr[1],drr[2], . . . ,drr[N]], (EQ. 17)

where the vectors drr[n] are:

drr[n]=[drr₁[n],drr₂[n], . . . ,drr_{M}[n]] (EQ. 18)

and drrⱼ[n]=rr₁[n]−rrⱼ[n]=(r[n]−r[n−1])−(r[n−j]−r[n−1−j]),j=1, . . . ,n−1

Any of the above statistical parameters can be selected for the analysis. All the selected parameters thus define a parameter space which is traversed by the method so as to determine the possible cardiac arrhythmia. Preferably the parameter space has five or more dimensions, which include at least the following statistical parameters: S^{rr}_{M}, S^{rbv}_{M}, VC^{rr}_{M}, VC^{rbv}_{M}, and std(drrs). In various exemplary embodiments of the invention the data are segmented to epoch segments, wherein each segment is represented as a vector within the parameter space. For example, when a five-dimensional parameters space is employed, five statistical parameters (e.g., S^{rr}_{M}, S^{rbv}_{M}, VC^{rr}_{M}, VC^{rbv}_{M}, and std(drrs)) are calculated for each segment, thereby representing the segment as a quintet forming a five-component vector. A typical duration of an epoch segment is from about 10 seconds to about 60 seconds.

Once the parameters are calculated, each segment is assessed for possible cardiac arrhythmia based on the calculated parameters for this segment. In some embodiments of the present invention, a thresholding procedure having a set of criteria is applied to the parameters. When the criteria are met, the method determines that a cardiac arrhythmia event is likely to be present within the respective epoch segment. A representative example of a thresholding procedure suitable for the present embodiments is provided in the Examples section that follows.

In some embodiments of the present invention the thresholding procedure is followed by an adaptive classification procedure in which the likelihood that a particular segment of the data indicates a cardiac arrhythmia event is determined. The adaptive classification procedure is preferably employed only for segments for which the criteria of the thresholding procedure are met. When the criteria of the thresholding procedure are not met, the adaptive classification procedure is preferably, but not necessarily, skipped. Thus the adaptive classification procedure serves as a second layer of classification. Embodiments in which the adaptive classification procedure is employed irrespectively of (e.g., without) the thresholding procedure are not excluded from the scope of the present invention.

An adaptive classification procedure according to some embodiments of the present invention will now be explained.

A localized mixture model is calculated for several segments, more preferably all segments, of the data, using the previously calculated statistical parameters of each segment. Thus, unlike the power density function in which the variables are direct observables of the data (e.g., time-intervals, blood volume values), the classification procedure uses the previously calculated parameters as the variables of the localized mixture model. In this respect, the classification procedure features a “second order” localized mixture model, since the model is constructed in the parameter space which by itself is constructed from direct observables of the data.

Optionally and preferably the second order localized mixture model is applied to all the statistical parameters. Thus, in this embodiment, the dimension of the model is the same as the dimension of the parameter space. A representative example for a localized mixture model suitable to be used as a second order mixture model is a Gaussian mixture model, but other types of mixture models (e.g., mixture models featuring a categorical distribution or a multinomial distribution or a Poisson distribution or an exponential distribution, or a multivariate Gaussian distribution or the like), are not excluded from the scope of the present invention.

A set of criteria is then applied to each localized function of the second order model, so as to provide a first model classified as normal and a second model classified as abnormal. For example, the collection of all localized functions for which the criteria are met can be defined as the abnormal model, and the collection of all localized functions for which one or more of the criteria is not met can be defined as the normal model. An abnormal model generally includes all localized functions corresponding to epoch segments that are suspected as having a cardiac arrhythmia event.

Once the two mixture models are defined, the likelihood that a particular segment has a cardiac arrhythmia event is determined by calculating the similarities of the segment to each of the two mixture models within the parameter space. When the similarity between the segment and the second model is higher than the similarity between the segment and the first model, the segment is marked as having a cardiac arrhythmia event. The similarities can be calculated by any technique. A representative example of such technique is the likelihood ratio test (LRT) [Kay, S. M. supra].

The method proceeds to 18 at which the method issues a report regarding the analysis. The report can include a list of epochs for which cardiac arrhythmia event it is likely to be present. The report can also includes unifications of two or more adjacent epochs for which cardiac arrhythmia event it is likely to be present. The report optionally and preferably also includes information pertaining premature heartbeats. Such information can be in the form of a list of time points at which a premature heartbeat has been identified, optionally with the type of premature heartbeat (e.g., premature atrial contraction, premature ventricular contraction). Alternatively or additionally, the information can include statistical quantities, such as the amount of identified premature heartbeat and/or the aggregated amount of time during which premature heartbeat have been identified. Optionally and preferably the information also includes the pattern associated with the identified premature heartbeat, e.g., ventricular bigeminy and ventricular trigeminy.

The method ends at 19.

Following is a description of a preferred technique for identifying premature heartbeats, according to some embodiments of the present invention. The identification procedure receives the ventricular depolarization features and calculates the pulse rate and duration between successive features. A premature heartbeat can then be determined by a thresholding procedure, wherein a pulse rate above a predetermined pulse rate threshold and a duration above a predetermined duration threshold indicate presence of a premature heartbeat. A typical value for the pulse rate threshold is 1.5-2.5 times the normal pulse rate and a typical value for the duration threshold is about 1 second.

When the data include PPG or continuous blood pressure data, additional criteria are preferably applied. These criteria can relate to the relative blood volume value.

According to some embodiments of the present invention the method compares the relative blood volume value to a first threshold V₁ wherein a relative blood volume value below the threshold indicates possible premature heartbeat. In some embodiments, the method compares the relative blood volume value of a feature to a second threshold V₂>V₁, wherein a relative blood volume value which is above V₂ indicates that the preceding feature possibly correspond to a premature heartbeat.

Also contemplated are embodiments in which the thresholds are selected to discriminate between premature ventricular contractions and premature atrial contractions. In these embodiments each feature is compared to four thresholds V₁, V₂, V₃ and V₄, where V₃<V₁ and V₄>V₂. If (i) the relative blood volume value of a feature n is below V₃ and (ii) the relative blood volume value of the feature n+1 is above V₄, then the method determines that the feature n corresponds to a premature ventricular contraction. If (i) the relative blood volume value of a feature n is between V₃ and V₁, and (ii) the relative blood volume value of the feature n+1 is between V₂ and V₄, then the method determines that the feature n corresponds to a premature atrial contraction.

The thresholds V₁, V₂, V₃ and V₄ can be predetermined or it can be selected adaptively. Typical values for these thresholds are, without limitation, V₁=50%, V₂=120%, V₃=20% and V₄=150%.

Alternatively or additionally, the pattern associated with the premature heartbeats (if exist can be estimated). These embodiments will now be explained in terms of the aforementioned time-intervals rr₁[n] and/or relative blood volume values rbv₁[n], wherein rr₁[n] is suitable for all types of data (e.g., ECG, PPG, continuous blood pressure), and rbv₁[n] is suitable for data indicative of blood volume (e.g., PPG data or continuous blood pressure data). In the present embodiments, a series of ratios between two successive time-intervals and/or a series of ratios between two successive relative blood volume values is calculated. The former series is denoted R_{T}[n] and can be written as R_{T}[n]=rr₁[n]/rr₁[n−1]. The latter series is denoted R_{V}[n] and can be written as R_{V}[n]=rbv₁[n]/rbv₁[n−1]. The series is/are preferably calculated for each feature in each segment. In various exemplary embodiments of the invention the dependence of R_{T} and/or R_{V} on n over each segment of the data is used for classifying the heartbeats. When R_{T} and/or R_{V} possess some periodicity over the segment, the method can determine that the segment includes premature ventricular contractions. In this case, the method can also estimate the pattern associated with the premature ventricular contractions. For example, when the periodicity of R_{T} and/or R_{V} is approximately 2 (two next-to-nearest neighbors elements in the series are similar to each other) the method can determine possible ventricular bigeminy, and when the periodicity of R_{T} and/or R_{V} is approximately 3 (two next-to-next-to-nearest neighbors elements in the series are similar to each other) the method can determine possible ventricular trigeminy.

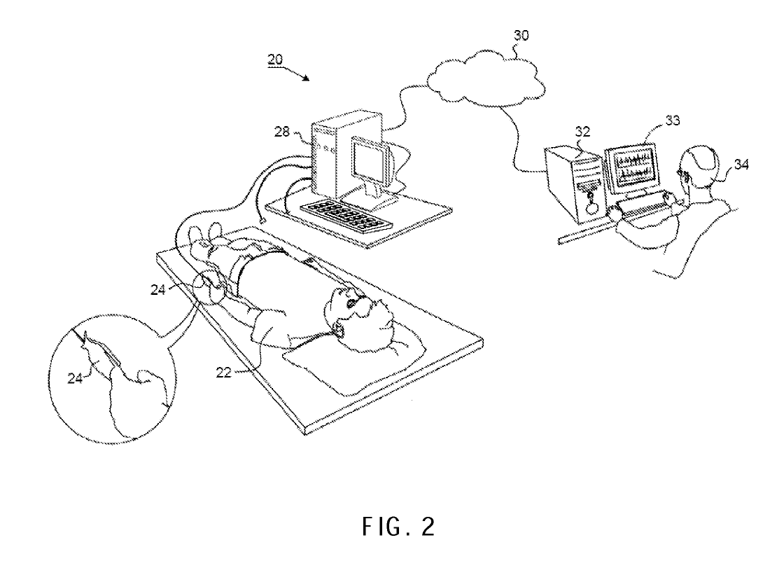

Reference is now made to FIG. 2 which is a schematic illustration of a system 20 for analyzing physiological data indicative of myocardial activity, according to some embodiments of the present invention. System 20 can be used is used for monitor a patient 22 in a home, clinic, hospital ward environment, or any other facility.

System 20 comprises a data processing system 28 configured for carrying out the method described above. Data processing system 28 can comprises a general-purpose computer processor (which may be embedded in a bedside or remote monitor) with suitable software for carrying out the method described above. This software may be downloaded to data processing system 28 in electronic form, or it may alternatively be provided on tangible media, such as optical, magnetic or non-volatile electronic memory. Alternatively, data processing system 28 can comprise a special computer processor configured for carrying out the functions described herein. For example, data processing system 28 can be a special computer processor which comprises special firmware embodying computer instructions for carrying out the functions described herein.

Data processing system 28 analyzes the data in order to identify in the data the ventricular depolarization features, calculate the time-intervals vector and/or relative blood volume values vector, fit the vector to a power density function and determine possible cardiac arrhythmia based on statistical parameters characterizing the power density function, as further detailed hereinabove. In some embodiments of the present invention data processing system 28 identifies data features corresponding to premature heartbeats, as further detailed hereinabove.

Optionally and preferably system 20 receives a signal indicative of the myocardial activity. Any of the aforementioned signals can be used. In the representative illustration of FIG. 2 the signal is a PPG signal received from a suitable sensor, such as a pulse oximetry device 24. Device 24 provides PPG signals indicative of blood flow and of the level of oxygen saturation in the patient's blood. In the context of the present patent application and in the claims, the PPG signal is thus considered to be a signal that is indicative of myocardial activity.

Additionally or alternatively, system 20 may comprise sensors of other types (not shown) and appropriate circuitry, for collecting other physiological signals. For example, the system may receive an ECG signal, measured by skin electrodes and/or continuous blood pressure sensors such as those commercially available under the tradename CNAP™. Sensors for continuous blood pressure monitoring can also include PPG sensors and/or electrical impedance plethysmograph sensors, as described, for example, in U.S. Pat. No. 6,413,223, the contents of which are hereby incorporated by reference. Sensors for continuous blood pressure monitoring can optionally include tonometric sensors as described, for example, in U.S. Pat. No. 5,165,416. Continuous blood pressure signals can also be acquired by system 20 via a combination of piezoelectric transducers and ultrasonic transducer, as described, for example, in U.S. Pat. No. 5,111,826.

The signals from device 24 and/or any other of the aforementioned and other sensors are collected, digitized and processed by data processing system 28. Data processing system 28 can receive the signals either directly from the sensors or by telemetry.

Data processing system 28 may process and analyze the signals from pulse oximetry device 24 locally, using the methods described hereinabove. Alternatively, data processing system 28 is coupled to communicate over a network 30, such as a telephone network or the Internet, with a remote data processing system 32. This configuration permits the analysis to be performed simultaneously in multiple different locations. In these embodiments remote data processing system 32 carries out the method described above.

The local 28 or remote 32 data processing system can output the results of the analysis to an operator 34, such as a physician, via an output device such as a display 33.

As used herein the term “about” or “approximately” refers to +/−10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

Embodiments of the present embodiments have been utilized to detect arrhythmias from ECG and PPG data

64 eligible patients were recruited from the Heart Failure Center at Lady Davis Carmel Medical Center/Lin Medical Center in Haifa, Israel. Patients were followed and treated for symptomatic HF of at least 1 month's duration. Treatment was according to AHA/ACC guidelines. Only patients willing to sign an informed consent were enrolled in the study. The study was approved by the medical center's Helsinki Committee. Included were 64 HF patients (54 men and 10 women; age range 27-88 years). The patient underwent full-night PSG sleep test, which include 12 leads ECG and PPG channel on top of the standard channels of PSG test i.e. EEG, chin EMG, EOG, LEG, oximetry, respiratory effort and respiratory flow.

Additional ECG data were obtained from Moody et al. supra and Goldberger et al. “PhysioBank, PhysioToolkit and PhsioNet: Components of a new Research Resource for Complex Physiologic Signals,” Circulation 2000, 101(23):e215-e220.

Some operations were implemented using epic [Bilms J., 1998, “A Gentle Tutorial of the EM Algorithm and its Application to Parameter Estimation for Gaussian Mixture and Hidden Markov Models,” ICSI TR-97-21] ANSI/AAMI standard episode by episode annotation comparator [Pudil et al., “Floating search methods in feature selection,” Pattern Recognition Letters, vol. 15, no. 11, pp. 1119-1125, 1994].

The data were preprocessed using a band pass filter (0.05-5 Hz) to suppress very low frequency and high frequency noise. The PPG data was detrended and the location and normalized area of each pulse in the PPG was detected. For each pulse, the duration from a previous peak and the ratio between successive peaks was calculated. Pulses passing a predetermined duration threshold (e.g., time-interval of more than 0.5 second) and a predetermined ratio threshold (e.g., above ratio of more than 0.5) were defined as ventricular depolarization features. The normalized area of each pulse was defined as a value indicative of relative blood volume.

The vectors rrs, drys and rbvs were calculated as described above. The rrs and rbvs vectors were used to constructs power density functions using a D-GMM. The heart rate was calculated from the PPG signal. Features corresponding to premature heartbeats were identified using the procedure described in Example 2, below, and bigeminy patterns and trigeminy patterns were discarded from further analysis.

FIGS. 3A-D show 10 seconds of normal heart rhythm. FIG. 3A shows an ECG signal during normal heart rhythm, and FIG. 3B shows the corresponding PPG signal. The number near each peak in the PPG signal is the calculated relative blood volume values. FIG. 3C shows the pulse rate of the PPG signal, and FIG. 3D shows a histogram of the vector rrs with M=10. As shown in FIG. 3D, there are exactly M typical distances occurring approximately every 1 second. This is due to the normal rhythm seen in FIGS. 3A and 3B.

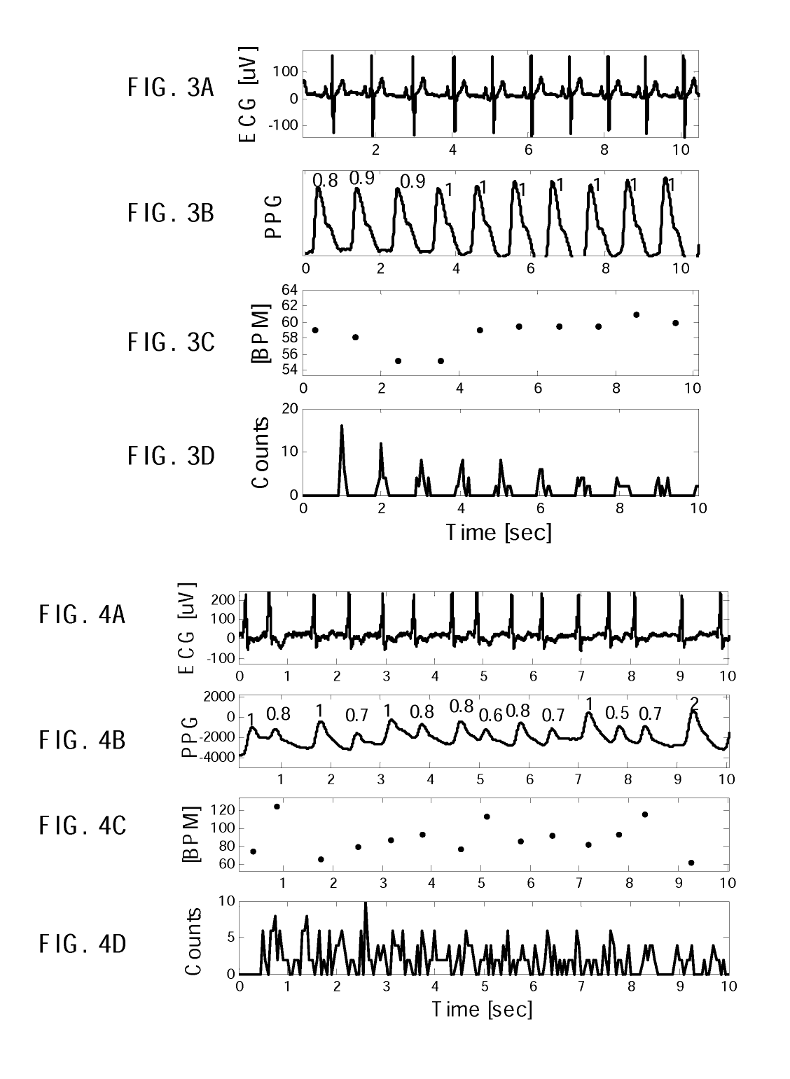

FIGS. 4A-D show 10 second of atrial fibrillation (AFIB) rhythm. FIG. 4A shows 10 seconds of ECG during AFIB rhythm, FIG. 4B shows the corresponding PPG signal and the calculated relative blood volume values, FIG. 4C shows the pulse rate of the PPG signal, and FIG. 4D shows a histogram of the vector rrs with M=10. As shown, the histogram of rrs during AFIB is chaotic and it is easily distinguished from a normal rhythm, see, e.g., the rrs histogram of normal rhythm shown in FIG. 3D.

The following procedure was employed for calculating the parameters λ, σᵢ and Δλᵢ in the vector θ. The expectation of the first Gaussian in rrs is the same as the expectation of rr₁[n]. Therefore, λ was obtained from rr₁[n] as the maximum location of its histogram. The fluctuations Δλ₁ and widths σᵢ were calculated using an observation vector Oᵢ where Oᵢ=rrs(x), xε[iλ−αλ, iλ+αλ], αε[0,1]. The widths σᵢ were calculated using the standard deviation of Oᵢ. For the fluctuations Δλᵢ the histogram of Oᵢ was obtained and the values of Δλᵢ were calculated using the argument of the maximum of the histogram. Formally, the following expressions were used: Δλᵢ=iλ+argmax(HIST(Oᵢ)|α=0.05), and σᵢ=std(Oᵢ|α=0.5), where HIST and std denote histogram and standard deviation, respectively. No re-estimation procedure was employed on iλ+Δλᵢ. Nevertheless, in some embodiments of the present invention the value of σᵢ is iterated by testing different values of α such that the power density functions are maximized.

Possible cardiac arrhythmia events were determined by traversing a five-dimensional parameter space which included the following statistical parameters: S^{rr}_{M}, S^{rbv}_{M}, VC^{rr}_{M}, VC^{rbv}_{M}, and std(drrs).

FIGS. 5A-C show normalized histograms of S^{rr}_{M}, VC^{rr}_{M} and std(drrs), respectively. Histograms of the parameters during AFIB are shown as dashed lines and Histograms of the parameters during normal rhythm are shown as solid lines. As shown, low values of S^{rr}_{M} correspond to AFIB, indicating overlap between Gaussians; low values of VC^{rr}_{M} correspond to normal rhythm whereas higher values of VC^{rr}_{M} correspond to AFIB rhythm; and low values of std(drrs) correspond to normal rhythm whereas higher values of std(drrs) correspond to AFIB rhythm. FIGS. 5A-C thus demonstrates the ability of the present embodiments to separate between AFIB and normal rhythms based on the calculated statistical parameters.

The determination of possible cardiac arrhythmia events included a thresholding procedure. For some segments an adaptive classification procedure was also employed.

The following criteria were used for the thresholding procedure. If the separation measure was smaller than 0.55 and the logarithm of the VC_{M} parameter was greater than −6.5 and the standard deviation of drrs was greater than 0.032 for more than 2 consecutive segments, then the underlying record was labeled as AFIB. Otherwise, the record was labeled as normal.

The adaptive classification procedure was employed only for records for which more than 5% but less than 95% of the segments were marked as AFIB.

The adaptive classification procedure featured a GMM which was calculated using an expectation maximization procedure for all the segments of the data. The Gaussians of the GMM were subjected to the same set of criteria as in the thresholding procedure. The Gaussians that were labeled AFIB were assembled to define an AFIB GMM, while Gaussians that were labeled normal were assembled to define a normal AFIB GMM. In order to decide whether a given segment is AFIB a likelihood ratio test was applied.

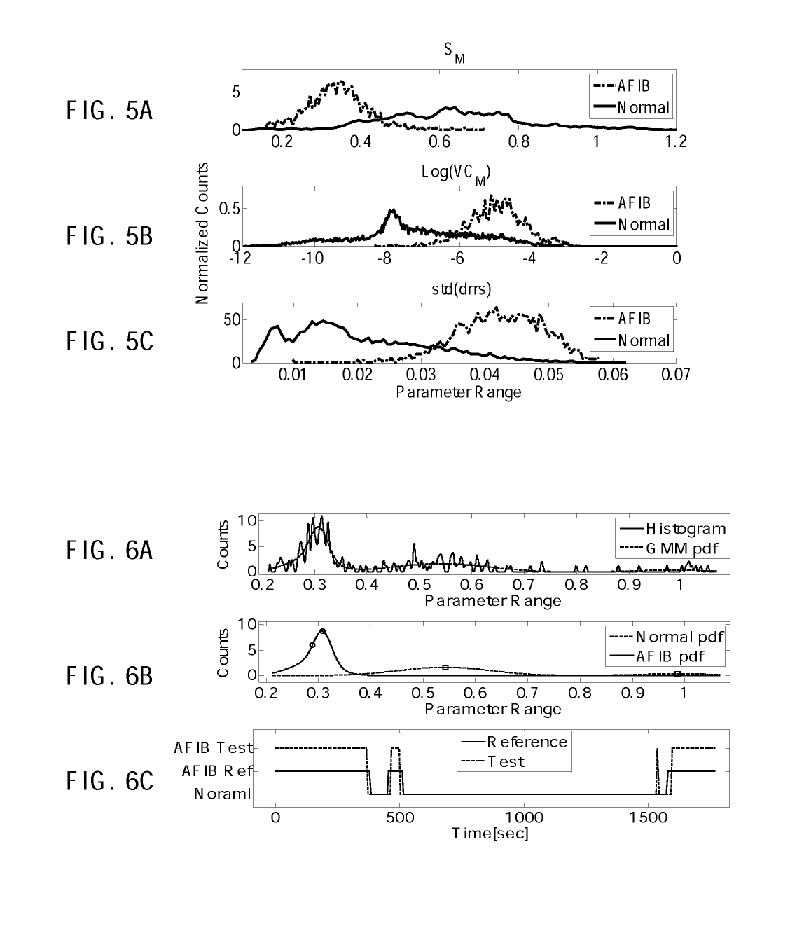

FIGS. 6A-C show the results of the adaptive classification procedure. FIG. 6A shows the normalized histogram (solid line) and the global GMM power density function (dashed line) estimated from the separation parameter extracted from record 201 of the MIT-BIH arrhythmia database. FIG. 6B shows the derivation of AFIB GMM and normal GMM from the global GMM by classifying the centers of the global GMM. The circles in FIG. 6B denote centers of the Gaussian of the global GMM that were classified as AFIB and the square denotes the centers of the Gaussian of the global GMM pdf that were classified as normal rhythms. FIG. 6C exemplifies the performance of the inventive technique using only the separation measure, were only one epoch of AFIB was mistakenly classified.

Table 1 below presents the performance of the present embodiments using 38 records of the MIT-BIH arrhythmia training database. A gross duration sensitivity of 94% and duration positive predictivity of 85% where achieved using the training data set. Table 2, below, presents the performance of the testing database which achieved a gross duration sensitivity and positive predictivity of 96%.

It is noted that 10 studies of the MIT-BIH arrhythmia database where included into the testing set in order to evaluate global classification performance. All records in training and testing set were correctly classified into AFIB or non AFIB records.

TABLE 1

| Record | TPs | FN | TPp | FP | ESe | E + P | DSe | D + P | Ref duration | Test duration |
|---|---|---|---|---|---|---|---|---|---|---|
| 111 | 0 | 0 | 0 | 0 | — | — | — | — | 0:00.000 | 0:00.000 |
| 112 | 0 | 0 | 0 | 0 | — | — | — | — | 0:00.000 | 0:00.000 |
| 113 | 0 | 0 | 0 | 0 | — | — | — | — | 0:00.000 | 0:00.000 |
| 114 | 0 | 0 | 0 | 0 | — | — | — | — | 0:00.000 | 0:00.000 |
| 115 | 0 | 0 | 0 | 0 | — | — | — | — | 0:00.000 | 0:00.000 |
| 116 | 0 | 0 | 0 | 0 | — | — | — | — | 0:00.000 | 0:00.000 |
| 117 | 0 | 0 | 0 | 0 | — | — | — | — | 0:00.000 | 0:00.000 |
| 118 | 0 | 0 | 0 | 0 | — | — | — | — | 0:00.000 | 0:00.000 |
| 119 | 0 | 0 | 0 | 0 | — | — | — | — | 0:00.000 | 0:00.000 |
| 121 | 0 | 0 | 0 | 0 | — | — | — | — | 0:00.000 | 0:00.000 |
| 122 | 0 | 0 | 0 | 0 | — | — | — | — | 0:00.000 | 0:00.000 |
| 123 | 0 | 0 | 0 | 0 | — | — | — | — | 0:00.000 | 0:00.000 |
| 124 | 0 | 0 | 0 | 0 | — | — | — | — | 0:00.000 | 0:00.000 |
| 200 | 0 | 0 | 0 | 0 | — | — | — | — | 0:00.000 | 0:00.000 |
| 201 | 3 | 0 | 3 | 1 | 100 | 75 | 99 | 82 | 10:05.800 | 12:12.000 |
| 202 | 4 | 0 | 3 | 1 | 100 | 75 | 91 | 81 | 9:46.313 | 11:00.311 |
| 203 | 19 | 1 | 3 | 0 | 95 | 100 | 98 | 86 | 21:32.405 | 24:18.780 |
| 205 | 0 | 0 | 0 | 0 | — | — | — | — | 0:00.000 | 0:00.000 |
| 207* | 0 | 0 | 0 | 0 | — | — | — | — | 0:00.000 | 0:00.000 |
| 208 | 0 | 0 | 0 | 0 | — | — | — | — | 0:00.000 | 0:00.000 |
| 209 | 0 | 0 | 0 | 0 | — | — | — | — | 0:00.000 | 0:00.000 |
| 210 | 8 | 1 | 4 | 0 | 89 | 100 | 98 | 98 | 29:29.919 | 29:23.111 |
| 212 | 0 | 0 | 0 | 0 | — | — | — | — | 0:00.000 | 0:00.000 |
| 213 | 0 | 0 | 0 | 0 | — | — | — | — | 0:00.000 | 0:00.000 |
| 214 | 0 | 0 | 0 | 0 | — | — | — | — | 0:00.000 | 0:00.000 |
| 215 | 0 | 0 | 0 | 0 | — | — | — | — | 0:00.000 | 0:00.000 |
| 217 | 10 | 14 | 1 | 0 | 42 | 100 | 58 | 77 | 4:12.433 | 3:12.000 |
| 219 | 7 | 3 | 5 | 0 | 70 | 100 | 86 | 93 | 23:46.711 | 22:00.000 |
| 220 | 0 | 0 | 0 | 0 | — | — | — | — | 0:00.000 | 0:00.000 |
| 221 | 12 | 0 | 3 | 0 | 100 | 100 | 98 | 98 | 29:16.563 | 29:18.852 |
| 222 | 33 | 0 | 6 | 0 | 100 | 100 | 94 | 41 | 8:32.550 | 19:27.169 |
| 223 | 0 | 0 | 0 | 0 | — | — | — | — | 0:00.000 | 0:00.000 |
| 228 | 0 | 0 | 0 | 0 | — | — | — | — | 0:00.000 | 0:00.000 |
| 230 | 0 | 0 | 0 | 0 | — | — | — | — | 0:00.000 | 0:00.000 |
| 231 | 0 | 0 | 0 | 0 | — | — | — | — | 0:00.000 | 0:00.000 |
| 232 | 0 | 0 | 0 | 0 | — | — | — | — | 0:00.000 | 0:00.000 |
| 233 | 0 | 0 | 0 | 0 | — | — | — | — | 0:00.000 | 0:00.000 |
| 234 | 0 | 0 | 0 | 0 | — | — | — | — | 0:00.000 | 0:00.000 |
| Sum | 96 | 19 | 28 | 2 | | | | | 2:16:42.694 | 2:30:52.223 |
| Gross | | | | | 83 | 93 | 94 | 85 | | |
| Average | | | | | 87 | 94 | 90 | 82 | | |

TABLE 2

| Record | TPs | FN | TPp | FP | ESe | E + P | DSe | D + P | Ref duration | Test duration |
|---|---|---|---|---|---|---|---|---|---|---|
| 04015 | 6 | 1 | 4 | 15 | 86 | 21 | 89 | 15 | 3:57.236 | 24:15.172 |
| 04043 | 77 | 5 | 80 | 6 | 94 | 93 | 89 | 81 | 2:11:59.704 | 2:24:59.492 |
| 04048 | 4 | 3 | 4 | 1 | 57 | 80 | 48 | 56 | 6:00.776 | 5:09.144 |
| 04126 | 5 | 2 | 6 | 0 | 71 | 100 | 90 | 95 | 22:57.972 | 21:44.504 |
| 04746 | 2 | 3 | 4 | 2 | 40 | 67 | 100 | 100 | 5:25:53.008 | 5:26:19.840 |
| 04908 | 8 | 0 | 7 | 23 | 100 | 23 | 94 | 62 | 51:22.452 | 1:18:22.936 |
| 04936 | 32 | 4 | 133 | 1 | 89 | 99 | 83 | 98 | 7:22:40.696 | 6:11:52.392 |
| 05091 | 1 | 7 | 1 | 7 | 13 | 13 | 49 | 10 | 1:26.768 | 6:53.184 |
| 05121 | 20 | 0 | 64 | 8 | 100 | 89 | 89 | 96 | 6:26:48.944 | 5:59:13.952 |
| 05261 | 2 | 9 | 2 | 2 | 18 | 50 | 75 | 76 | 7:59.492 | 7:53.476 |
| 06426 | 26 | 0 | 100 | 0 | 100 | 100 | 96 | 96 | 9:45:13.536 | 9:46:35.572 |
| 06453 | 3 | 3 | 4 | 0 | 50 | 100 | 55 | 85 | 6:11.280 | 4:00.364 |
| 06995 | 4 | 2 | 38 | 5 | 67 | 88 | 95 | 98 | 4:49:17.516 | 4:40:02.036 |
| 07162 | 1 | 0 | 37 | 0 | 100 | 100 | 98 | 100 | 10:13:42.344 | 10:03:35.444 |
| 07859 | 1 | 0 | 23 | 0 | 100 | 100 | 100 | 100 | 10:13:42.868 | 10:10:57.692 |
| 07879 | 1 | 1 | 52 | 0 | 50 | 100 | 97 | 100 | 6:10:02.688 | 5:57:21.968 |
| 07910 | 4 | 1 | 4 | 2 | 80 | 67 | 99 | 97 | 1:37:49.220 | 1:39:21.448 |
| 08215 | 2 | 0 | 28 | 1 | 100 | 97 | 99 | 100 | 8:14:33.968 | 8:09:21.976 |
| 08219 | 38 | 1 | 47 | 28 | 97 | 63 | 94 | 65 | 2:12:28.852 | 3:12:02.944 |
| 08378 | 5 | 0 | 19 | 1 | 100 | 95 | 94 | 99 | 2:08:18.408 | 2:02:17.924 |
| 08405 | 2 | 0 | 12 | 0 | 100 | 100 | 100 | 100 | 7:23:09.528 | 7:22:24.984 |
| 08434 | 3 | 0 | 3 | 3 | 100 | 50 | 99 | 88 | 23:43.736 | 26:49.616 |
| 08455 | 2 | 0 | 12 | 1 | 100 | 92 | 96 | 100 | 7:04:31.024 | 6:49:24.000 |
| 100 | 0 | 0 | 0 | 0 | — | — | — | — | 0:00.000 | 0:00.000 |
| 101 | 0 | 0 | 0 | 0 | — | — | — | — | 0:00.000 | 0:00.000 |
| 102 | 0 | 0 | 0 | 0 | — | — | — | — | 0:00.000 | 0:00.000 |
| 103 | 0 | 0 | 0 | 0 | — | — | — | — | 0:00.000 | 0:00.000 |
| 104 | 0 | 0 | 0 | 0 | — | — | — | — | 0:00.000 | 0:00.000 |
| 105 | 0 | 0 | 0 | 0 | — | — | — | — | 0:00.000 | 0:00.000 |
| 106 | 0 | 0 | 0 | 0 | — | — | — | — | 0:00.000 | 0:00.000 |
| 107 | 0 | 0 | 0 | 0 | — | — | — | — | 0:00.000 | 0:00.000 |
| 108 | 0 | 0 | 0 | 0 | — | — | — | — | 0:00.000 | 0:00.000 |
| 109 | 0 | 0 | 0 | 0 | — | — | — | — | 0:00.000 | 0:00.000 |
| Sum | 249 | 42 | 684 | 106 | | | | | 93:23:52.016 | 92:51:00.060 |
| Gross | | | | | 86 | 87 | 96 | 96 | | |
| Average | | | | | 79 | 78 | 88 | 83 | | |

### Example 2

Embodiments of the present embodiments have been utilized to detect premature heartbeats. ECG and PPG data were obtained, preprocessed and the ventricular depolarization were identified as described in Example 1 above.

The series R_{T}[n] and R_{V}[n] were calculated from rr₁[n] and rbv₁[n] as described above.

FIGS. 8A-B shows a representative example of a premature ventricular contraction beat as it is manifested on the ECG (FIG. 8A) and the PPG (FIG. 8B). As shown in both figures, the QRS of the premature ventricular contraction is wide and has no existing P wave. Additionally, the premature ventricular contraction has prolonged compensatory pause before the next heartbeat appears. As shown in FIG. 8B, the relative blood volume value of the premature ventricular contraction is 0.1, the relative blood volume of a normal beat (e.g., a beat preceding the premature ventricular contraction) is 1, and the relative blood volume of the beat immediately following the compensatory pause is twice the size of a normal beat. Thus, a first relative blood volume threshold V₁ of about 50% and a second relative blood volume threshold V₂ of 120% can be used according to some embodiments of the present invention for identifying the premature ventricular contraction and the immediately following beat, respectively.

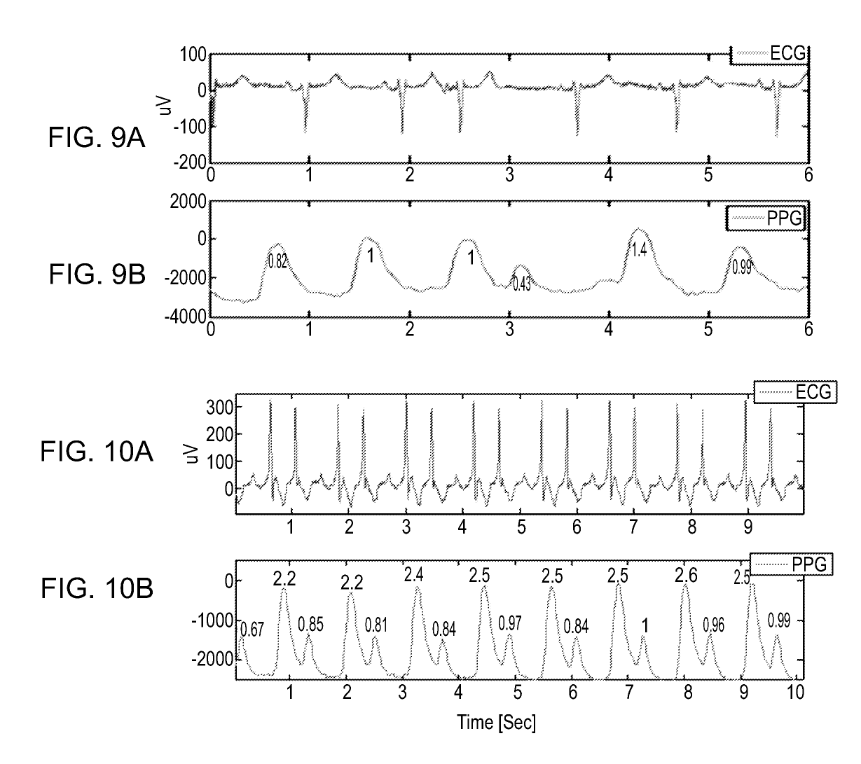

FIGS. 9A-B show a premature atrial contraction as manifested on the ECG (FIG. 9A) the PPG (FIG. 9B). As shown in FIG. 9A, the P wave of the premature atrial contraction overlaps with the T wave of the preceding heartbeat. Also shown is the prolonged compensatory pause following the premature contraction. As shown in FIG. 9B the relative blood volume of the premature atrial contraction beat is lower than the relative blood volume of the normal beat but is higher than the relative blood volume of a premature ventricular contraction. The relative blood volume of the heartbeat immediately following the compensatory pause after a premature atrial contraction is higher than the relative blood volume of a normal beat but lower than the relative blood volume of a heartbeat following the compensatory pause of a premature ventricular contraction. Thus, a third relative blood volume threshold V₃ of about 20% and a fourth relative blood volume threshold V₄ of 150% can be used according to some embodiments of the present invention for discriminating between premature ventricular contraction and premature atrial contraction.

FIGS. 10A-B show bigeminy rhythm as manifested on the ECG (FIG. 10A) and PPG (FIG. 10B). As shows, there is a periodicity of 2 both in the ECG and in the PPG. Thus, periodicity in the series R_{T} or R_{V} as defined above can be used for identifying pattern associated with the premature ventricular contractions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of analyzing physiological data indicative of myocardial activity, comprising:
identifying in the data a set of N features, each corresponding to a ventricular depolarization, N being an integer larger than 1;
for each ventricular depolarization feature, calculating M time-intervals, thereby providing a vector of N*M time-intervals, M being an integer larger than 1;
fitting said vector to a power density function of time-intervals; and
determining possible cardiac arrhythmia based on statistical parameters characterizing said power density function of said time-intervals.

2. The method according to claim 1, wherein said power density function comprises a sum of localized functions.

3. The method according to claim 2, wherein an expectation and variance of each of said localized functions are independent of a time-point of a respective ventricular depolarization feature.

4. The method according to claim 2, wherein said power density function represents a degenerated mixture model.

5. The method according to claim 4, wherein said degenerated mixture model is a degenerated Gaussian mixture model.

6. The method according to claim 1, wherein said parameters comprise at least one of a time-interval separation parameter, and a variance of ratios between widths and centers characterizing said power density function of said time-intervals.

7. The method according to claim 1, further comprising calculating, for each time-interval, M differences between time-intervals, thereby providing a vector of N*M time-interval differences, wherein said possible cardiac arrhythmia is determined based, at least in part, on a standard deviation of said vector of time-interval differences.

8. The method according to claim 1, further comprising, for each ventricular depolarization feature, calculating at least one relative blood volume value, thereby providing a vector of at least N relative blood volume values, and fitting said vector of relative blood volume values to a power density function of relative blood volume values;
wherein said determination of possible cardiac arrhythmia is also based on an additional set of statistical parameters characterizing said power density function of said relative blood volume values.

9. (canceled)

10. (canceled)

11. The method according to claim 1, further comprising applying to said statistical parameters a thresholding procedure having a set of criteria, wherein fulfillment of said criteria by said statistical parameters indicates possible arrhythmia among said N ventricular depolarization features.

12. The method according to claim 1, wherein if said criteria are fulfilled, then the method further comprises:
calculating a second order localized mixture model within a space spanned by said statistical parameters;
applying a set of criteria to said second order localized mixture model so as to provide a model classified as normal and a model classified as abnormal;
for a given segment of the data, determining the likelihood that said segment is abnormal by calculating similarities of said segment to said first and said second models.

13. The method according to claim 12, wherein each of said localized mixture models is independently a Gaussian mixture model.

14. The method according to claim 1, wherein said data comprise photoplethysmograph data.

15. The method according to claim 1, wherein said data comprise electrocardiogram data.

16. The method according to claim 1, wherein said data comprise continuous blood pressure data.

17. The method according to claim 1, further comprising identifying data features corresponding to premature heartbeats.

18. Computer-readable medium having stored thereon a computer program, wherein said computer program comprising code means that when executed by a data processing system carry out the method according to claim 1.

19. A system for analyzing physiological data indicative of myocardial activity, comprising a data processing system configured for:
identifying in the data a set of N features, each corresponding to a ventricular depolarization, N being an integer larger than 1;
for each ventricular depolarization feature, calculating M time-intervals, thereby providing a vector of N*M time-intervals, M being an integer larger than 1;
fitting said vector to a power density function of time-intervals; and
determining possible cardiac arrhythmia based on statistical parameters characterizing said power density function of said time-intervals.

20. The system according to claim 19, wherein said power density function comprises a sum of localized functions.

21. The system according to claim 20, wherein an expectation and variance of each of said localized functions are independent of a time-point of a respective ventricular depolarization feature.

22. The system according to claim 20, wherein said power density function represents a degenerated mixture model.

23. The system according to claim 22, wherein said degenerated mixture model is a degenerated Gaussian mixture model.

24. The system according to claim 19, wherein said parameters comprise at least one of a time-interval separation parameter, and a variance of ratios between widths and centers characterizing said power density function of said time-intervals.

25. The system according to claim 19, wherein said data processing system is configured for calculating, for each time-interval, M differences between time-intervals, thereby providing a vector of N*M time-interval differences, wherein said data processing system is configured to determine said possible cardiac arrhythmia based, at least in part, on a standard deviation of said vector of time-interval differences.

26. The system according to claim 19, wherein said data processing system is configured for calculating, for each ventricular depolarization feature, at least one relative blood volume value, thereby providing a vector of at least N relative blood volume values, and fitting said vector of relative blood volume values to a power density function of relative blood volume values;
wherein said determination of possible cardiac arrhythmia is also based on an additional set of statistical parameters characterizing said power density function of said relative blood volume values.

27. (canceled)

28. (canceled)

29. The system according to claim 19, wherein said data processing system is configured for applying to said statistical parameters a thresholding procedure having a set of criteria, wherein fulfillment of said criteria by said statistical parameters indicates possible arrhythmia among said N ventricular depolarization features.

30. The system according to claim 19, wherein said data processing system is configured for:
calculating a second order localized mixture model within a space spanned by said statistical parameters;
applying a set of criteria to said second order localized mixture model so as to provide a model classified as normal and a model classified as abnormal;
for a given segment of the data, determining that the likelihood that said segment is abnormal by calculating similarities of said segment to said first and said second models.

31. The system according to claim 30, wherein each of said localized mixture models is independently a Gaussian mixture model.

32. The system according to claim 19, wherein said data comprise photoplethysmograph data.

33. The system according to claim 19, wherein said data comprise electrocardiogram data.

34. The system according to claim 19, wherein said data comprise continuous blood pressure data.

35. The method according to claim 19, wherein said data processing system is configured for identifying data features corresponding to premature heartbeats.

36. The system according to claim 19, further comprising at least one sensor for receiving a signal indicative of said myocardial activity, and circuitry for generating said physiological data responsively to said signal.

37. The system according to claim 36, wherein said at least one sensor comprises at least one of: a photoplethysmograph, an electrocardiogram lead and blood pressure sensor.

38. (canceled)

39. (canceled)
